(19) United States
(12) Patent Application Publication
(10) Pub. No.: US 2017/0143236 A1
(43) Pub. Date: May 25, 2017

(54) SYSTEMS AND METHODS FOR OPTICALLY GUIDED PLACEMENT AND MONITORING OF MEDICAL IMPLANTS

(71) Applicants: Children's National Medical Center, Washington, DC (US); Virginia Tech Intellectual Properties, Inc, Blacksburg, VA (US)

(72) Inventors: Raj Shekhar, Dayton, MD (US); Mahdi Azizian, Santa Clara, CA (US); Peng Cheng, Fairfax, VA (US); Lawrence Mahan, Bethesda, MD (US); Abby Whittington, Christiansburg, VA (US); Lissett Bickford, Blacksburg, VA (US)

(73) Assignee: Children's National Medical Center, Washington, DC (US)

(21) Appl. No.: 15/421,954

(22) Filed: Feb. 1, 2017

Related U.S. Application Data
(63) Continuation of application No. 14/493,137, filed on Sep. 22, 2014.
(60) Provisional application No. 61/883,318, filed on Sep. 27, 2013.
(60) Provisional application No. 61/880,538, filed on Sep. 20, 2013.

(51) Int. Cl. A61B 5/06; A61M 25/01; A61B 5/00

(52) U.S. Cl. CPC A61B 5/066; A61B 5/0095; A61B 5/0035; A61B 5/064; A61M 25/0105; A61M 2240/00

(57) ABSTRACT

Described herein are systems and corresponding methods to place and further monitor an implanted medical device. The implanted device includes a fluorescent material that is disposed on a portion of a tip of the device. The system also includes a skin patch having one or more infrared light detectors configured to detect light radiation from the fluorescent material on the implanted device located beneath a skin surface of living tissue. The system further includes an image processing module that is configured to construct an image of the implanted device and its surroundings. The processor further registers and analyzes the position of the implanted device and provides an appropriate feedback signal to a monitoring station.

### CROSS-REFERENCE TO RELATED APPLICATIONS

This application is based upon and claims the benefit of priority under 35 U.S.C. §119(e) from Provisional U.S. Patent Application Ser. No. 61/880,538, filed on Sep. 20, 2013, and Provisional U.S. Patent Application Ser. No. 61/883,318, filed Sep. 27, 2013, the entire contents of each of which are herein incorporated by reference.

### BACKGROUND

Field of the Invention

The present disclosure is related to optical imaging of implanted medical devices with a goal of guiding an initial placement of the medical device and further ensuring a continued proper placement of the device after implantation. Specifically, the present disclosure describes a fluorescence-based optical imaging system and related methods thereof for periodic monitoring of peripherally inserted central catheters and other medical implant devices of similar nature.

Related Art

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Advances in medicine have led to several implantable devices. A frequently used implant in both children and adults is peripherally-inserted central catheters (PICCs), or as commonly referred to, PICC lines. PICC lines are long flexible catheters that are inserted into a peripheral vein, typically in the upper arm, and advanced until the catheter tip rests just outside of the heart, frequently in the distal superior vena cava or cavoatrial junction. The PICC lines remain in situ for extended periods of time, often ranging from a few days to a few months, and provide a mechanism for administering nutrition, blood, and medication, and can be further used for blood sampling purposes. The long-term placement of PICC lines increases the likelihood of their migration from a desired position due to factors such as body movement, body growth and the like. The improper placement or the migration of the PICC lines from the desired position can have adverse effects such as vascular perforation (pierced blood vessel), venous thrombosis (blocked blood vessel), and pericardial tamponade (pressure on the heart), all of which can have fatal consequences. In addition to PICC migration, the insertion of the PICC can be difficult and often requires multiple adjustments in order for the tip of the catheter to be correctly placed. For instance, studies have revealed that only approximately 66% of PICC lines are inserted correctly the first time, and around 2 to 10.5% of PICC lines dislodge throughout the course of implantation.

Accordingly, the placement and monitoring of the implanted PICC lines is crucial. However, the methods typically implemented for addressing the monitoring and placement problems of the PICC lines have severe limitations. For instance, X-rays are commonly used to confirm the final placement of the catheter tip or to refine the placement if not positioned appropriately. In the specific case of PICC lines, the tip of the catheter can be seen against anatomical structures, such as the ribs. However, neonates are particularly at an increased risk from prolonged radiation exposure involved in X-ray imaging, including proclivity to develop lymphoma and other forms of cancer at a later stage of their life. To minimize radiation, X-ray-based monitoring is used infrequently, often weekly or biweekly. However, the PICC line may migrate between two such monitoring events, thereby causing serious complications.

Another method for monitoring PICC lines is ultrasound. While ultrasound is useful in PICC line placement, it has limited utility in monitoring the implanted PICC line because the catheter is not easily visualized in ultrasound. In neonates, trans-illumination with visible light is commonly used to guide PICC line insertion, but this method also has limitations in optimally visualizing the vasculature. A PICC line cannot be visualized using trans-illumination as visible light has limited penetration in the human body. Due to these reasons, trans-illumination cannot be used as a viable technique for periodic monitoring of the implanted PICC lines.

A newer and still evolving technique uses hemolytic and electrocardiography data to calculate whether the placement of the PICC line tip is correct. The method does not provide a physician with the much-needed visual image of the tip and the surrounding vasculature. Furthermore, due to the large diameter of PICC lines, this method is feasible to be implemented only in adults. Children and neonatal babies have small body sizes and thus are not ideal candidates on whom this method can be implemented. The method also does not help with monitoring after implantation.

Another proposed method passes red light (high wavelength visible light) through a modified fiber-optic stylet to guide PICC line placement. This method in its current form also cannot assist with monitoring due to limited penetration of visible light into the human tissue.

Accordingly, there is a medical requirement for imaging implanted medical devices such as a catheter without the use of ionizing radiation in order to avoid inherent risks, and further develop an efficient technique of placing and monitoring of the PICC lines.

### SUMMARY

The present disclosure provides for methods that can image and monitor modified peripherally inserted central catheters and other medical devices within the body. The disclosure provides for an imaging configuration that images implanted devices inside living tissue at certain working depths. Further, embodiments described herein provide for the long-term monitoring of the implanted device through frequent examination of the device's position inside the body through a process varying from manual to a completely autonomous one. Specifically, a fluorescence molecular imaging technique in the near-infrared region to view implants either with a coating of fluorescent dye or made with polymeric materials impregnated with a fluorescent dye is presented.

Further, embodiments described herein provide for implanted devices such as peripherally inserted central catheters (fluorescent-coated or made with fluorescent-impregnated material), to be imaged during placement for proper insertion. The imaging technique can also be used for imaging of cardiac implants, joint surfaces, and endotracheal tubes. Furthermore, after initial placement of the implanted device, an optical imaging system such as a vein viewing system or a second imaging modality such as ultrasonography can produce an image of the tissues surrounding the implanted device. The imaging techniques described herein are noninvasive and the resulting constructed images can be integrated with the captured fluorescence image to provide a complete visualization of the implanted device along with its surrounding tissues.

Additionally, embodiments described herein provide for an adjustable skin patch that includes a plurality of sensors. The skin patch is configured to interact with the implanted device and transmit a signal to indicate whether the implanted device has migrated from an intended position.

Thus, according to one embodiment there is provided a medical device monitoring system. The system includes a medical device having applied thereon a near infrared (NIR) dye and positioned under a skin of a patient, a patch containing boundary markers positioned on the skin of the patient, an NIR emitter that emits NIR light that reacts with the NIR dye and boundary markers, an imager configured to construct an image of the medical device and the patch based on infrared light received from the medical device and the boundary markers, and an image processor configured to detect and register, based on the constructed image, a relative location of the boundary markers and the medical device.

According to one embodiment there is provided a patch positioned on the skin of a patient. The patch includes a plurality of near infrared (NIR) transmitters disposed on the patch, each transmitter configured to emit NIR light that reacts with a NIR dye disposed on a medical device that is positioned under the skin of the patient, a plurality of NIR detectors configured to receive NIR light emitted by the NIR dye, and circuitry configured to construct an image of the medical device based on the NIR light received by the detectors, and detect and register, based on the constructed image, a location of the medical device relative to a boundary of the skin patch.

According to one embodiment there is provided a method of tracking a location of an implanted medical device. The method includes the steps of stimulating, by an infrared transmitter, the implanted medical device having applied thereon a near infrared (NIR) dye and a patch containing boundary markers positioned on the skin of a patient, constructing an image of the implanted medical device and the patch based on infrared light transmitted by the medical device and the boundary markers, and detecting and registering by an image processor, based on the constructed image, a relative location of the boundary markers and the implanted medical device.

According to one embodiment there is provided an imaging device for inserting a medical device in a patient. The imaging device includes an imager configured to image a vein of the patient in which the medical device having applied thereon a near infrared (NIR) dye is to be inserted, and construct an image of the medical device being inserted in the vein, based on NIR light emitted by the NIR dye. Also included is circuitry configured compute a change in intensity of the NIR light emitted by the dye, and detect and register a position of the medical device relative to the vein based on the computed intensity change.

According to one embodiment there is provided a medical device monitoring system. The system includes a medical device having applied thereon a near infrared (NIR) dye and positioned under a skin of a patient, an emitter that emits high energy intensity light that reacts with the NIR dye and high intensity light that excites tissues surrounding the medical device, a photoacoustic and gray-level ultrasound imager configured to construct an image of the medical device and the locations around the medical device from distortions generated as a result of the reaction with the NIR dye and the excitation of the tissues surrounding the medical device, and an image processor configured to detect and register, based on the constructed image, a relative location of the boundary markers and the medical device.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 depicts an optical window suitable for biological imaging;

FIG. 2 depicts an exemplary implant device including a fluorescent dye coating and an implant device containing a composite material that includes the fluorescent dye;

FIGS. 3A-3F illustrates a skin patch including multiple near infrared marker patterns according to one embodiment;

FIG. 4 illustrates an exemplary fabrication process for composite catheters;

FIG. 5 depicts an imaging system (imager) according to one embodiment;

FIG. 6 is an exemplary flow diagram illustrating the workflow for monitoring peripherally inserted central catheter;

FIG. 7 illustrates an exemplary top view of a skin patch used in autonomous monitoring of catheters;

FIG. 8 illustrates according to an embodiment, a workflow for placement and monitoring of peripherally inserted central catheters;

FIG. 9A illustrates a catheter modified with a dye according to one embodiment;

FIG. 9B illustrates a near infrared signal from an implanted catheter according to another embodiment;

FIG. 9C illustrates a signal detected by fluorophores covered with muscular tissue according to one embodiment;

FIG. 10 is an exemplary flow diagram of a method for determining a location of an implanted catheter according to one embodiment;

FIG. 11 is an exemplary computing system for an image processor according to one embodiment;

FIG. 12 illustrates according to an embodiment, the thermal degradation heating profiles of dye (IRDye 800CW) and thermoplastic polyurethane (TPU);

FIG. 13 illustrates a computer aided design schematic of an annular dye;

FIG. 14 depicts exemplary optical images and scanning electron microscopy images of hollow polymer samples;

FIG. 15 illustrates exemplary atomic force microscopy (AFM) three dimensional micrographs of TPU tube samples;

FIG. 16 illustrates according to an embodiment, a graph depicting the mechanical properties of catheters;

FIG. 17 illustrates according to an embodiment, retention analysis of an IR Dye 800 CW within a TPU matrix according to an embodiment;

FIG. 18 depicts a graph illustrating stability of the IR Dye 800 CW in phosphate buffered saline powder;

FIG. 19 depicts an exemplary computer aided design schematic of annular dye;

FIG. 20 depicts an exemplary fluorescent intensity scan of Plain TPU, TPU Composite, and Leached TPU composite;

FIG. 21 is a graph depicting contrast enhancement intensity factor of TPU composites;

FIG. 22 depicts biocompatibility of thin films according to an embodiment; and

FIG. 23 illustrates adhesion of Human Umbilical Vein Endothelial cells on top of substrates;

### DETAILED DESCRIPTION

Advances in optical imaging are enabling many new applications and capabilities. Optical imaging techniques can scan the body tissues at reasonable depths and without any harmful effects. One such technique, fluorescence imaging, holds potential for many applications.

Fluorescence, which is a form of luminescence, is an imaging technique that uses the light emitted from an excited substance to create an image. Specifically, fluorophores (light-producing molecules), when excited by light of an appropriate wavelength, emits light of a longer wavelength (lower energy), which can be detected by a sensor or a camera system (e.g. charge-coupled devices). Fluorophores are available in a variety of excitation or emission wavelengths. However, the human body is most transparent to light penetration in the near-infrared (NIR) range of approximately 650 nm to 950 nm. Selecting a fluorescent dye containing fluorophores active in this region of the electromagnetic spectrum allows for optimal penetration of light into the body tissue. Therefore, NIR imaging is an appropriate imaging technique for visualizing PICC lines and other medical implants at certain penetration depths within the human body.

Peripherally inserted central catheters are hollow polymeric tubes that transport nutrients, blood and medications to neonates. NIR polymer composites can be fabricated into the PICCs by incorporating a fluorescent dye (IR Dye 800 CW, for example) and further visualized using NIR imaging. In order to fabricate the PICCs, polymer and dye are dry mixed and pressed, sectioned, and extruded to produce hollow tubes.

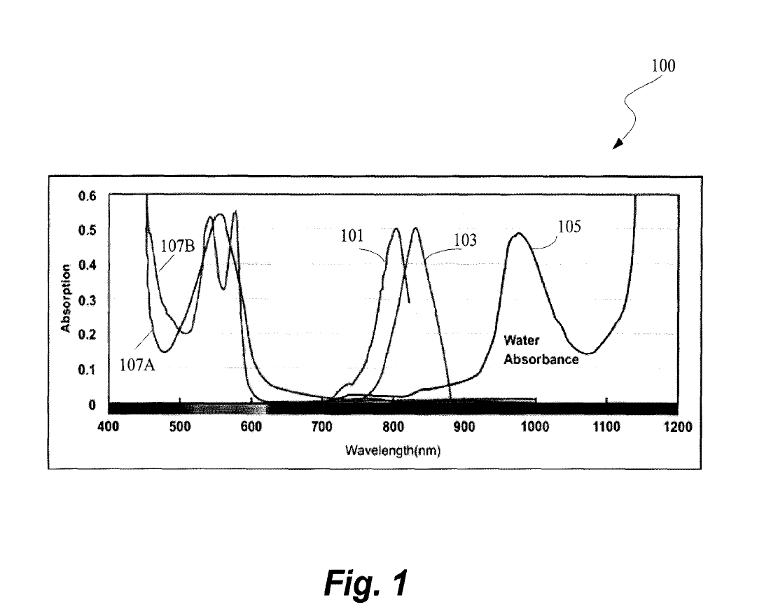

FIG. 1 depicts an optical window 100 suitable for biological imaging. Near-infrared light coinciding with this window passes through the human body with the least amount of absorption and scattering from blood and water. According to one embodiment, the use of a fluorescence dye with excitation and emission peaks within this window is used in NIR imaging,

The main tissue components that absorb light are hemoglobin and melanin which have high absorption bands at wavelengths shorter than 600 nm. As shown in FIG. 1, curves 107A and 107B correspond to the absorption of light by Oxy-hemoglobin and Deoxy-hemoglobin, respectively. Further, curve 105 represents the amount of absorption incurred by water. Water begins to absorb significant amounts of light at wavelengths above 1150 nm. Thus, there is a window (between ˜650 nm-950 nm) where biological tissue components do not absorb significant light, thus allowing imaging at depths ranging from 2-4 cm. Specifically, as shown in FIG. 1, curves 101 and 103 represent the excitation spectra and emission spectra of NIR light that can be employed for imaging purposes. For sake of convenience, in the remainder of the disclosure, the terms PICC and catheter are used synonymously to imply an implant device.

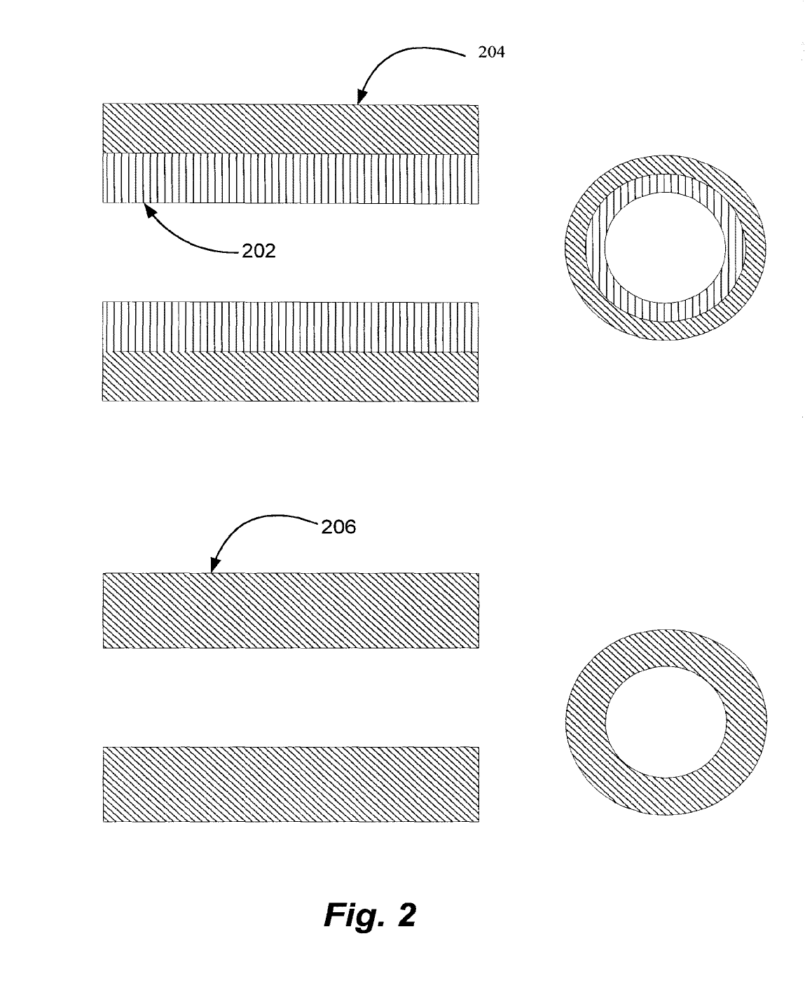

FIG. 2 depicts an exemplary implant device including a fluorescent dye coating and an implant device containing a composite material that includes the fluorescent dye. The fluorescent tip of a catheter can take two different forms. A first type of fluorescent tip has a catheter material 202 with a fluorescent dye coating 204. A second type of fluorescent tip has a polymeric material which contains a fluorescent dye matrix or composite 206. The pattern of coating can be adapted to the needs of the underlying applications. In one embodiment, only predetermined fraction of length of the catheter tip may be made to fluoresce.

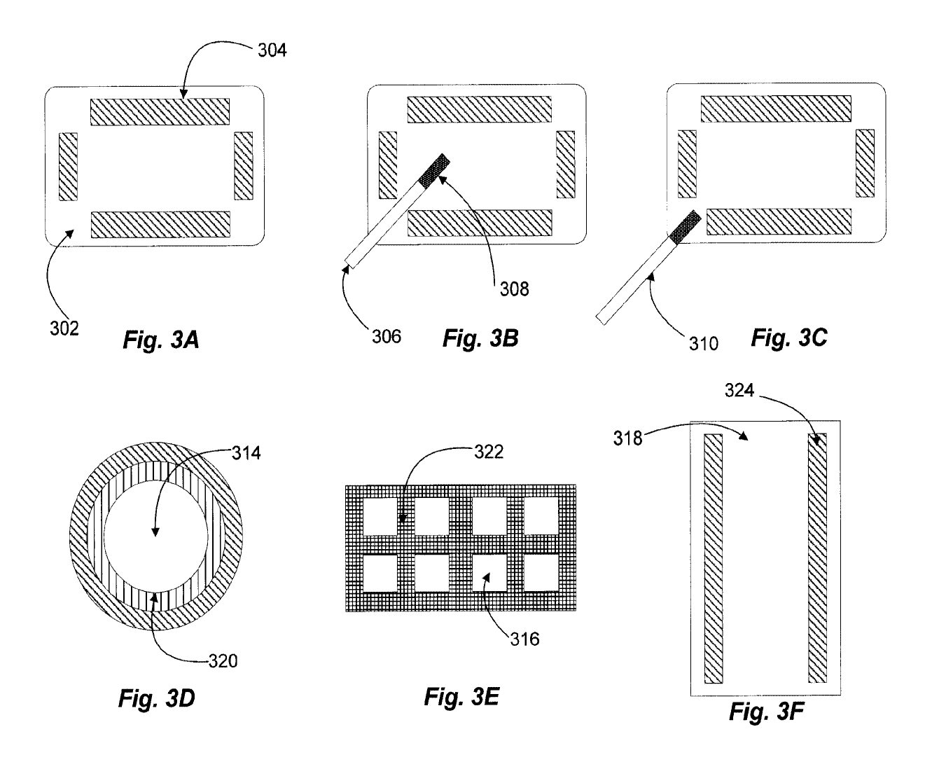

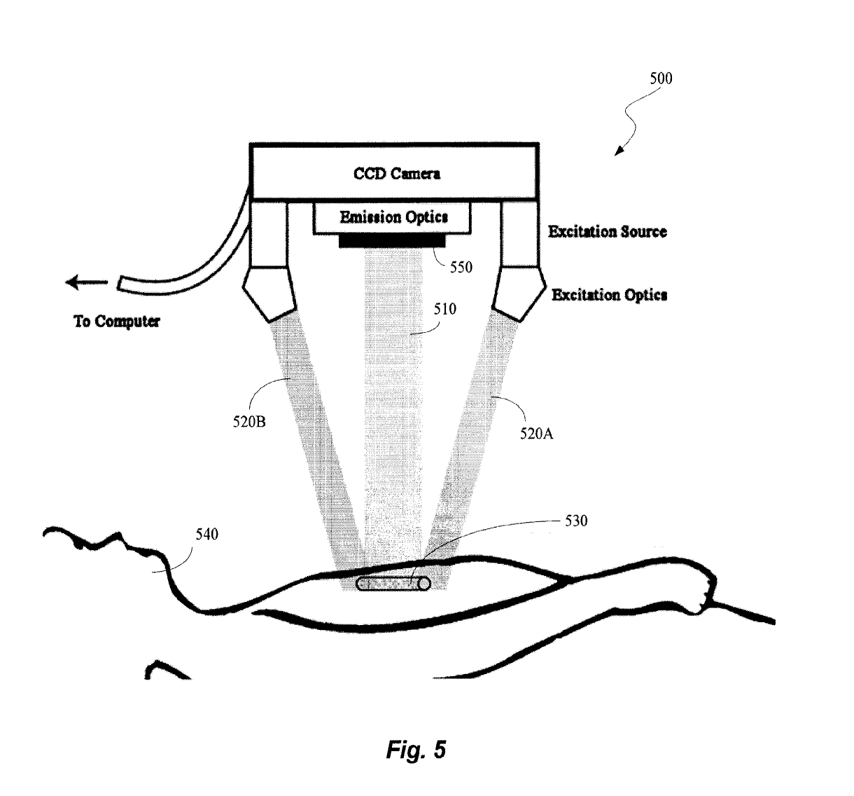

FIGS. 3A-3F illustrates a skin patch including multiple infrared marker patterns according to one embodiment. FIG. 3A illustrates a monitoring skin patch 302 that is used to monitor a PICC line or other type of implanted medical device containing a NIR fluorescent dye. The monitoring skin patch 302 comprises fluorescent markers made of near-infrared fluorophores 304. An imaging system (described with reference to FIG. 5) detects the light emitted from the fluorescent markers and the implanted device in order to determine if the implanted device is within a user-defined safe range.

FIG. 3B illustrates the catheter 306 is in a safe position, wherein the fluorescent tip 308 is within the safe range, as the tip is within the boundary marked by the fluorescent markers 304. FIG. 3C depicts a scenario wherein the catheter 310 is detected outside of the perimeter delimited by the fluorescent markers. In such a case, the imaging system (that processes the light received from the catheter and the markers) may be configured to generate and transmit an alarm signal to one or more monitors and/or health provider monitoring stations, indicating that a migration of the catheter tip has occurred.

The skin patch 302 is placed on the patient with its center approximately coinciding with the location where the implanted device of interest is initially placed. Other less preferred embodiments include splitting the patch into a number of independent patches and replacing or augmenting the patch with markers that are implanted or marked on or in the skin. However, by providing a single patch on the skin provides significant advantages such as ensuring that the markers are at a constant distance from one another.

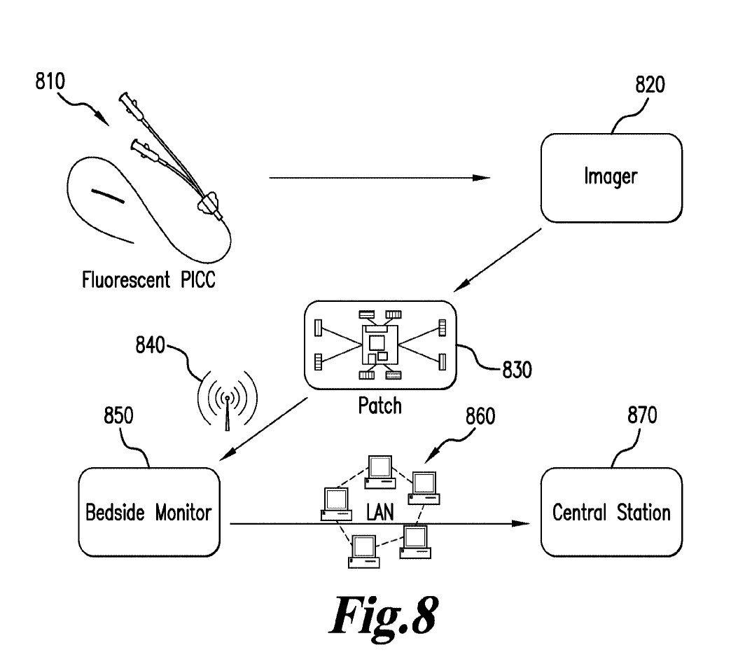

According to an embodiment, imaging techniques can also be used to determine a placement of the catheter. Such a catheter placement is described later with reference to FIG. 8. Specifically, a fluorescent PICC line can be monitored with imaging guidance provided by an imager. When the PICC line is in place and its tip location is confirmed with an augmented image obtained from another imaging system such as a vein viewer, ultrasound, or x-ray, the skin patch 302 can be applied to the subject's skin.

The skin patch 302 can be secured to the skin of the patient with glue, suture, or tape. The skin patch 302 can be square, oval, round 314, rectangular 316, or polygonal 318 as illustrated in FIGS. 3D, 3E, and 3F. The NIR fluorescent markers on the patch are placed in the shape of a circle 320, grid 322, or discrete rectangles 324 as illustrated in FIGS. 3D, 3E, and 3F. Note that the embodiments described herein are for illustrative purposes only, and are not intended to limit the scope of the present disclosure to include any combination of patch shapes or marker placements. Furthermore, the embodiments described herein use NIR fluorescence imaging principles to monitor implanted medical devices. The systems and methods monitor the location of implanted PICC lines by detection of fluorescence and by using automated image processing.

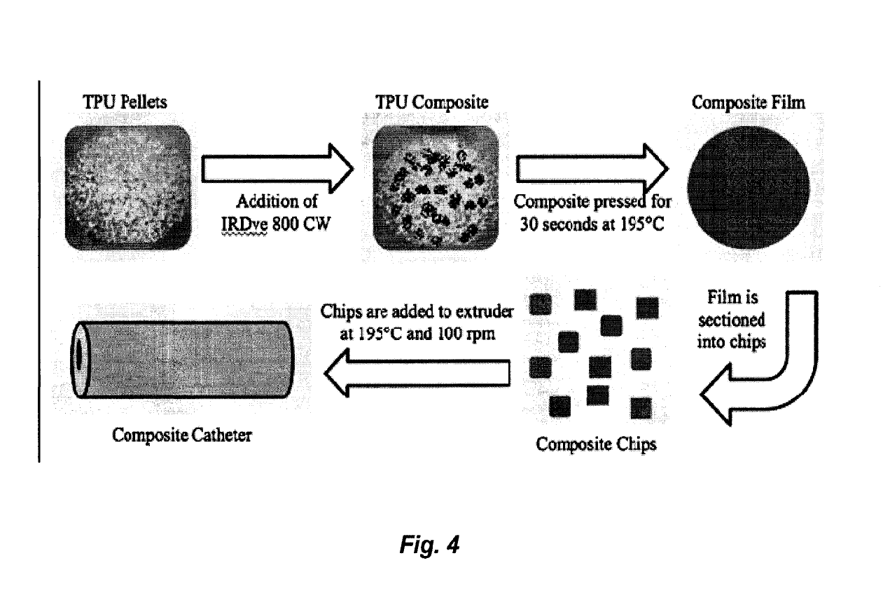

FIG. 4 illustrates an exemplary fabrication process for composite catheters. According to an embodiment, thin film thermoplastic polyurethane (TPU) pellets with and without IR-Dye 800CW (i.e., TPU Composite and Plain TPU) are fabricated. As shown in FIG. 4, the fabrication includes addition of the IR-Dye 800CW to plain TPU pellets, which are pressed for a predetermined amount of time to form a composite film. For instance, 5 grams of TPU with 0.025 wt % IRDye 800CW is pressed for 30 seconds and further sectioned into 5 mm squares, which are eventually fed into a compounder (e.g., a Haake Minilab Micro Compounder) to generate the composite catheter. Note that the catheters are extruded at 100 rpm at 195° C. using a custom die fabricated via an additive. Moreover, the extruded sections of Plain TPU and TPU Composites can be imaged and outer diameter measurements can be obtained using calipers. In addition, inner diameter measurements can be obtained using scanning electron microscopy (SEM), and the thickness measurements of the catheter can be calculated by subtracting the inner radius from the outer radius.

FIG. 5 depicts an imaging system 500 according to one embodiment. As shown in FIG. 5, the imager includes four modules: excitation light source, excitation optics, emission optics, and a CCD camera. First and foremost, fluorescent molecules in the medical implant need to be excited by a light source before they emit a signal. The present embodiment includes an excitation source in the form of a high power laser, laser diode, light-emitting diodes or the like. The excitation source is set to emit at a certain wavelength in the near-infrared band of the electromagnetic wave spectrum in order to allow for maximum light penetration into the tissue.

Excitation optics are properly calibrated to allow passing of the light of only a desired wavelength. The excitation optics include an optional diffuser to spread the light beam and a collimator to orient the resulting beam into a preferred direction, thereby resulting in a wider excited area. Specifically, light beams 520A and 520B are light beams that are incident on the catheter 530 that is implanted into a patient 540. Upon excitation of the fluorescence tip of the catheter, the beam 510 is emitted to the imager. In other words, after excitation of the catheter, the fluorophores embedded in the catheter emit a light at a longer wavelength that is incident on a filter 550 that is placed before the emission optics.

The filter 550 prevents unnecessary wavelengths of light from reaching the CCD camera that may interfere with image reconstruction. The CCD camera captures fluorescence light that is modified by emission optics, such as a lens, to enhance the signal for further processing. The captured light by the CCD camera converts photons into measureable electrical signals to create an image of the fluorescent device and the surrounding anatomy.

The imaging system as depicted in FIG. 5 is non-invasive. Specifically, the system does not require infiltration into the human body. Thus, the imager of the present embodiment allows for the processing of a generally cleaner and safer mode of visualization. Additionally, the imager is made to be portable, wherein the imager can be mounted and stationed on a cart with a moveable arm allowing for steady and simple holding. Yet according to another embodiment, the imager is a compact handheld imager incorporating all the modules described above. The imager can also be used to guide the insertion of a PICC line into a peripheral vein while continuously visualizing the PICC line. Specifically, with additional vein viewing capability, the corresponding vein can be visualized together with the PICC line.

A fluorescent PICC line can be implanted with imaging guidance provided by the imager into the body of a recipient. The imager can be implemented as a catheter viewer and a vein viewer as one device that alternates rapidly between imaging the catheter and the vein in which the catheter must be placed. The two images can be presented as a single image to the user. Ultrasound imaging can also be used to image veins at larger depths. In addition, a drop or increase in NIR signal intensity can give information about relative changes in depth and therefore detect if the device migrates from the intended position.

The device can display to the user a location of the PICC line with respect to the position of the vein to enable the user to see both the vein and the fluorescently marked catheter thereby providing the user with guidance for inserting the catheter. In addition, feedback can be provided to the user to ensure that the catheter is being properly inserted.

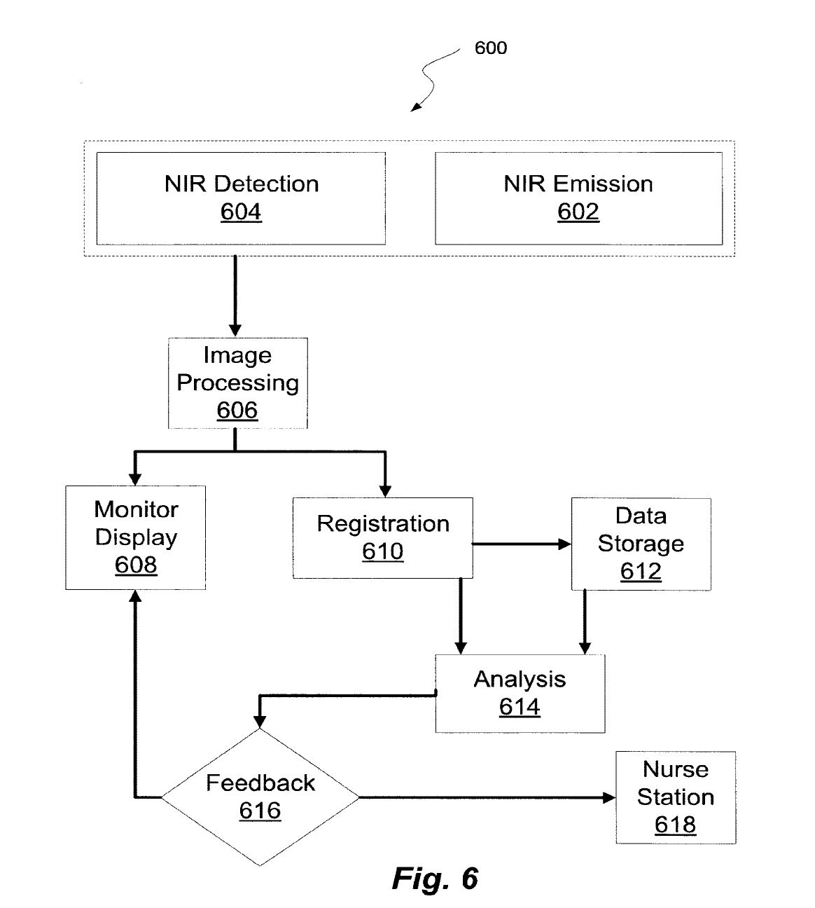

FIG. 6 depicts an exemplary flow diagram 600 illustrating the workflow for monitoring peripherally inserted central catheters. The imager includes an excitation light source (an NIR emission module) 602 and a NIR detector 604, such as a CCD camera. Fluorescent molecules present in the medical implant are excited so as to emit a signal. The excitation light source 602 can be a high power laser, laser diode, or light-emitting diode. The excitation light source 602 is configured to emit a wavelength in the NIR band of the electromagnetic wave spectrum, as described in FIG. 1, in order to allow for maximum light penetration into the tissue. Excitation optics are calibrated to allow passing of the light of the desired wavelength. The excitation optics can use an optional diffuser to spread the beam and a collimator to orient the resulting beam into a preferred direction.

After excitation, the fluorophores embedded in the medical device emit light at a longer wavelength. A filter placed before the emission optics prevents unnecessary wavelengths of light from reaching the CCD camera. The resulting light is modified by emission optics for further processing and analysis. Emission optics contain an emission filter to block out interfering light and a lens to enhance the signal. The resulting light illuminates a CCD camera system that converts photons into measureable electrical signals to create an image of the fluorescent device.

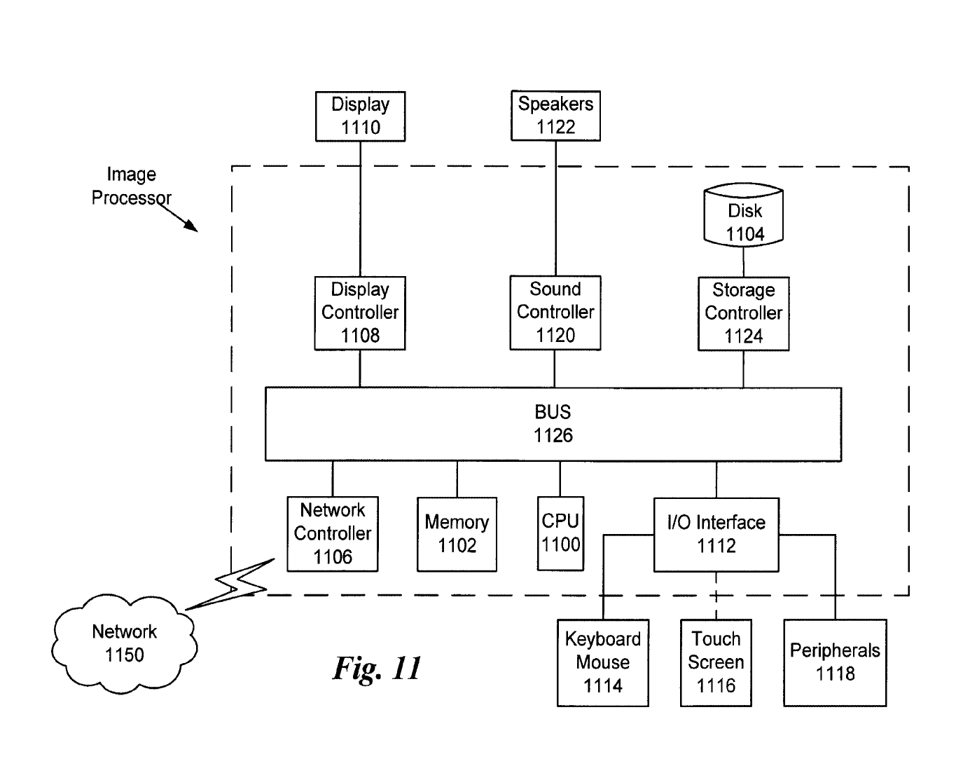

The NIR emission module 602 excites the fluorescent molecules on the markers located on the skin patch and the catheter tip. The fluorescent markers are also made of NIR fluorophores. The signal emitted by the fluorophores is detected by the NIR detection module 604. The signal is then processed by an image processing module 606 and consequently fed to a monitor display 608 and a registration module 610. The image processing module 606 includes a processor/processing circuitry that is configured to signal processing computations on the received NIR light signal. Details of the processor are described later with reference to FIG. 11. Further, the position of the fluorescent catheter tip and the fluorescent markers on the patch are registered in the registered module 610 and analyzed in the analyzer 614 with their last recorded positions stored in data storage 612. The image processing module 606 is configured to determine whether the position of the catheter is within pre-defined boundaries and/or whether the catheter is at its last determined position, and accordingly generate a feedback 616 to display the image of the catheter and the surrounding tissue on the display monitor of a care provider 608. Alternatively, if the position of the catheter has migrated considerably from its initial position, for example the catheter has migrated outside the boundaries delimited by the markers (on the skin patch), a signal is generated to be transmitted to a care provider station/monitor 608 and/or to a remote station 618.

Alternatively, according to another embodiment, a user-defined safe range could be established to determine how much the implanted device deviates from its original position, thereby indicating that the implanted device requires further inspection and/or adjustment. For instance, a predetermined deviation threshold could be established and further the shift in position of the catheter from its initial (or last determined) position can be computed to determine if the magnitude in shift of the catheter position is greater than the predetermined threshold. According to another embodiment, the image processing module of the imager may be configured to determine if the location of the catheter is determined to be within a certain predetermined distance away from the boundaries of the markers. In doing so, the imager is configured to detect in advance that the catheter has deviated considerably from its initial position and is approaching the boundary of the marker. Thus, a precautionary signal may be transmitted to the station 618 in order to notify the imminent risk that may be incurred due to the position of the catheter being considerably deviated. Furthermore, the preset boundaries of the safe range (zone) can be based upon the type of implanted device and parameters corresponding to individual recipients, such as the recipient's age, size, and pre-diagnosed medical conditions of the recipient.

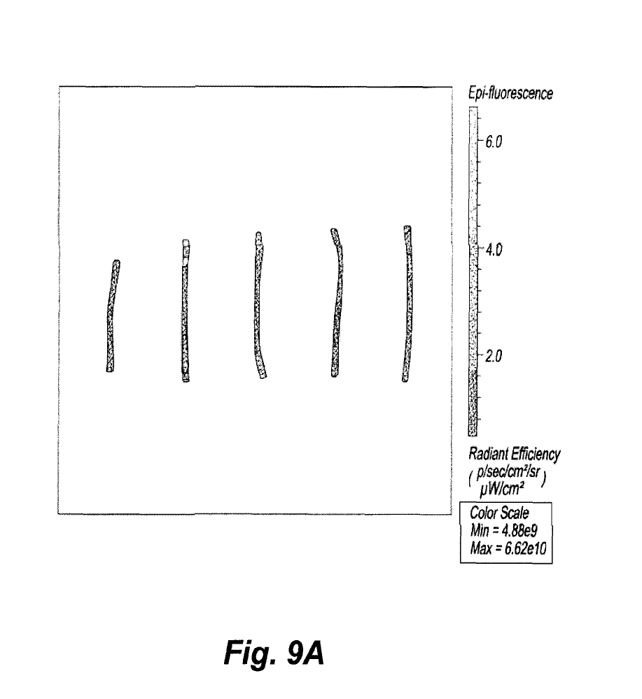

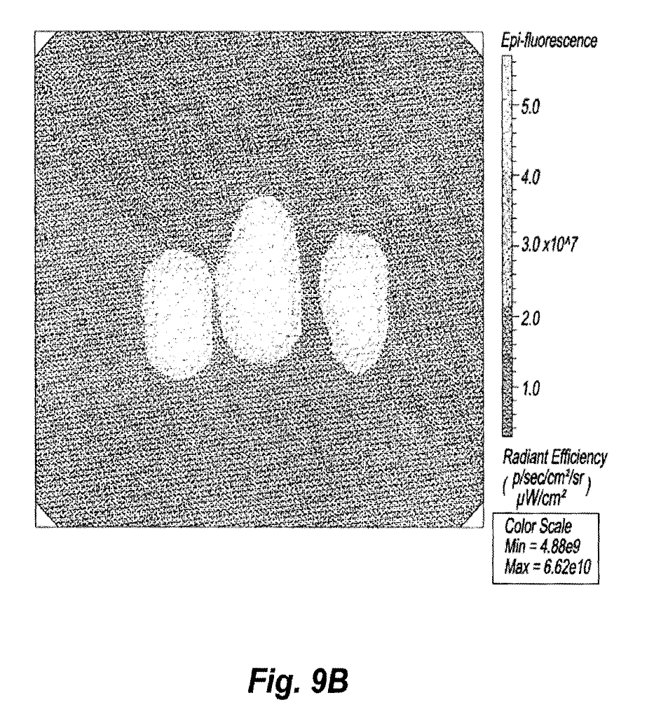

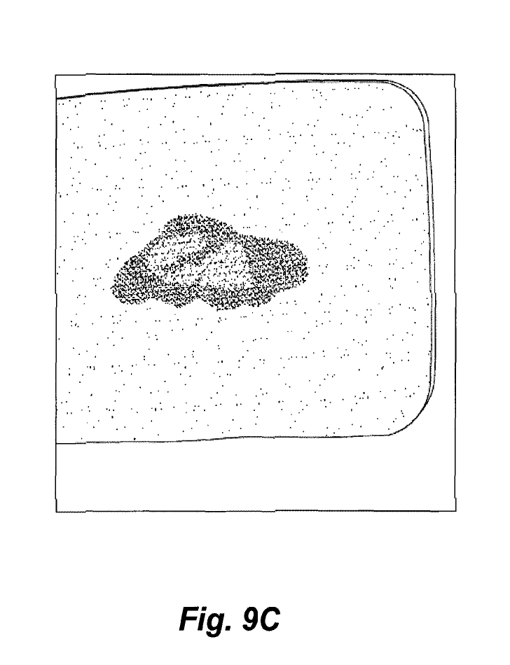

According to another embodiment, the catheter can be modified with a dye ALEXA FLUOR 680. FIG. 9A depicts an NIR signal constructed from such a catheter. Additionally, when the catheter is covered with approximately 1.9 cm of porcine muscular tissue, the same catheter exhibits an intense NIR signal, which provides a specific catheter location as illustrated in FIG. 9B. Alternatively, the fluorophores IRDYE 800CW can also be employed with similar effects as illustrated in FIG. 9C. Furthermore, NIR fluorescent dyes can include, but are not limited to, the list of NIR fluorescent dyes listed in Table 1 below.

TABLE 1
List of NIR fluorescent dyes used in implanted medical devices.

| NIR Dye name | Absorption Max (nm) | Emission Max (nm) |
|---|---|---|
| ICG | 780 | 810 |
| ALEXA FLUOR 680 | 679 | 702 |
| ATTO 700 | 699 | 720 |
| ALEXA FLUOR 790 | 778 | 808 |
| CF 790 | 784 | 811 |
| DYLIGHT 800 | 771 | 798 |
| IRDYE 800CW | 776 | 800 |
| CY7 NHS | 750 | 773 |

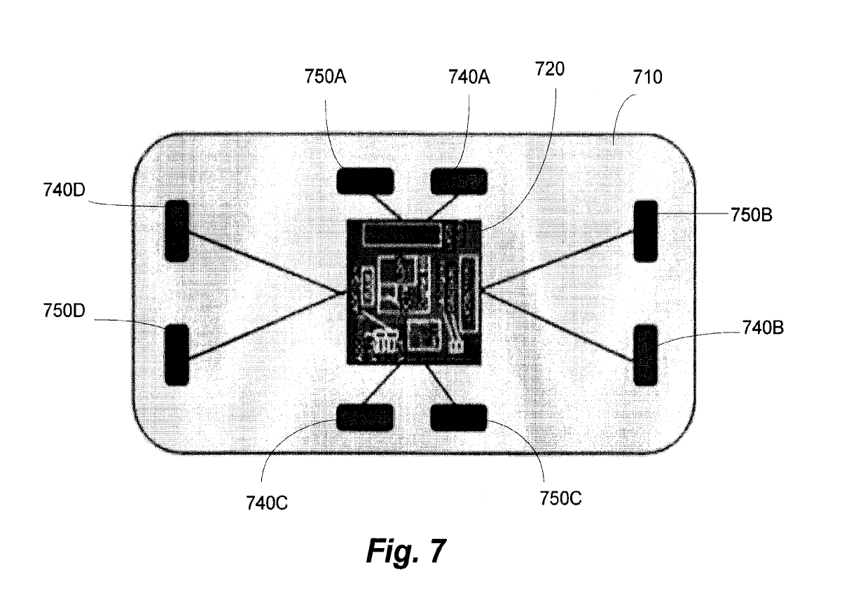

FIG. 7 illustrates an exemplary top view of a skin patch 710 used in autonomous monitoring of catheters. The monitoring of catheters is performed by an imager-assisted implanted PICC line. Specifically, the skin patch 710 includes near infrared transmitters 740A-740D that are disposed in a predetermined fashion on the surface of the patch. The transmitters are configured to transmit near infrared light in order to excite the fluorescence catheter. The light emitted by the fluorescence catheter is captured by detectors 750A-750D and further processed to generate an electrical signal that is processed by a processor 720 that is also disposed on the surface of the skin patch. The processor 720 is configured to perform the functions of the image processing module described with reference to FIG. 6.

In this manner, the skin patch 710 is configured to detect the emitted light from the catheter and determine if the catheter is within a safe region with respect to markers that are also present on the surface of the skin patch. The processor 720 is further configured to transmit a signal wirelessly (or alternatively in a wired fashion) to a remote terminal for monitoring and display purposes.

FIG. 8 illustrates according to an embodiment, a workflow for placement and monitoring of peripherally inserted catheters. Initially, a fluorescent PICC line 810 is implanted with imaging guidance provided by the imager 820 into the body of a recipient. Once the PICC line 810 is in place and its tip location is confirmed with an augmented image from another imaging system such as a vein viewer or ultrasound or even an x-ray, the skin patch as described in FIG. 8, can be applied to the recipient's skin. Subsequently, the patch can monitor the catheter tip periodically or even continuously to ensure that it has not deviated from its original intended location. If the tip migrates, the patch is configured to transmit a signal to a bedside monitor 850, which, in turn, can relay the alert signal to a central station 870 that is monitored by personnel. Furthermore, the central station 870 may be connected to the bedside monitor 850 by a local area network 860. Therefore, the workflow as depicted in FIG. 8 aids in the placement and monitoring of catheter like devices.

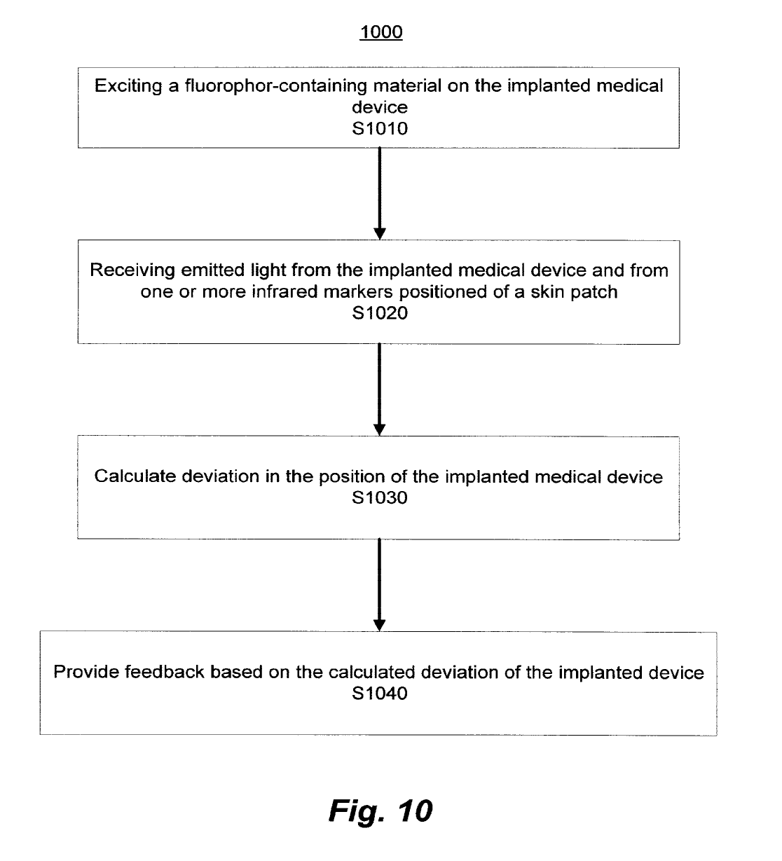

FIG. 10 is an exemplary flowchart, illustrating a method 1000 for determining a location of an implanted medical device such as a catheter. The medical device could also be a central venous catheter, dialysis catheter, drainage device, feeding device, imaging device, implantable port device, Interventional Radiology (I.R.) PICC, midline catheter, nursing PICC, port access needle, procedural accessory, stabilization device among others. The present embodiments can also be used for the confirmation of endotracheal tube placement, feeding tube placement and monitoring, and central venous line catheter placement in adults, umbilical access catheters, among others.

In step S1010, a fluorophor-containing material that is disposed on the tip of the catheter is excited by a near infrared light source. According to an embodiment, the near infrared light source may an external excitation light source (as described in FIG. 5) or alternatively, the light source may be embedded within a skin patch (as described in FIG. 7) that is positioned on a recipient's skin. Additionally, the near infrared light also excites markers that are positioned on a skin patch and which are made of a fluorophor material similar to that as the tip of the catheter.

In step S1020, upon receiving the infrared light from the light source, the fluorophor containing material on the catheter tip and infrared markers that are positioned on the skin path, emit light of a higher wavelength. The emitted light from the catheter and the markers is detected by detection device such as a CCD camera, as described in FIG. 5. Alternatively, according to another embodiment, a plurality of detectors may be disposed on skin patch as described in FIG. 7. The received light is processed by an image processor (as described in FIGS. 5 and 6) to generate an image of the implanted device and its surroundings by using near infrared image reconstruction techniques.

In step S1030, the image processor computes the deviation in the position of the implanted device. According to one embodiment, a deviation or shift in the position of the implanted device can be computed based on previously reconstructed images of the implanted devices. Furthermore, the image processor is also configured to determine whether the catheter is positioned in a safe zone based on the boundaries delimited by the markers on the skin patch.

The process upon computing the deviation of the implanted medical device in step S1030 proceeds to step S1040. In step S1040, the image processor is configured to provide a feedback notification. According to an embodiment, the notification may be based on the magnitude of deviation of the implanted device. For instance, if the magnitude of deviation is greater than a predetermined threshold, a notification (in the form of an alarm signal) may be transmitted to a monitoring station indicating that an adjustment of the implanted device is required. Alternatively, if the magnitude of deviation is minimal, a feedback notification to only display the image of the catheter may be performed. Furthermore, it must be appreciated that the notification signals may include a combination of alarm and display signals (or their equivalents).

Further, the method 1000 can also include filtering undesirable wavelengths of light that are emitted from the one or more infrared markers and the fluorophor tip of the medical device.

The medical device monitoring system can include a PICC line implanted device. The fluorescent material can contain fluorophores active in a near infrared electromagnetic spectrum range. The portion of the implanted device can contain a fluorescent dye coating or it can contain a fluorescent dye composite. The implanted device can also be configured to image a joint surface, a cardiac region, or an endotracheal tube.

The excitation source can include a high power laser, a laser diode, or one or more light-emitting diodes. The imager can also include excitation optics that is configured to restrict passage of an electromagnetic wave spectrum to a near infrared band only. The imager can also include a camera system that is configured to convert received photons into measurable electrical signals to create an image of the implanted device. The imager can be a mounted and portable device or a compact handheld device.

A hardware description of the image processor according to exemplary embodiments is described with reference to FIG. 11. In FIG. 11, the image processor includes a CPU 1100 which performs the processes described above. The process data and instructions may be stored in memory 1102. These processes and instructions may also be stored on a storage medium disk 1104 such as a hard drive (HDD) or portable storage medium or may be stored remotely. Further, the claimed embodiments are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the image processor communicates, such as a server or computer.

Further, the claimed embodiments may be provided as a utility application, background daemon, or component of an operating system, or combination thereof, executing in conjunction with CPU 1100 and an operating system such as Microsoft Windows 7, UNIX, Solaris, LINUX, Apple MAC-OS and other systems known to those skilled in the art.

CPU 1100 may be a Xenon or Core processor from Intel of America or an Opteron processor from AMD of America, or may be other processor types that would be recognized by one of ordinary skill in the art. Alternatively, the CPU 1100 may be implemented on an FPGA, ASIC, PLD or using discrete logic circuits, as one of ordinary skill in the art would recognize. Further, CPU 1100 may be implemented as multiple processors cooperatively working in parallel to perform the instructions of the inventive processes described above.

The image processor in FIG. 11 also includes a network controller 1106, such as an Intel Ethernet PRO network interface card from Intel Corporation of America, for interfacing with network 1150. As can be appreciated, the network 1150 can be a public network, such as the Internet, or a private network such as an LAN or WAN network, or any combination thereof and can also include PSTN or ISDN sub-networks. The network 1150 can also be wired, such as an Ethernet network, or can be wireless such as a cellular network including EDGE, 3G and 4G wireless cellular systems. The wireless network can also be WiFi, Bluetooth, or any other wireless form of communication that is known.

The image processor further includes a display controller 1108, such as a NVIDIA GeForce GTX or Quadro graphics adaptor from NVIDIA Corporation of America for interfacing with display 1110, such as a Hewlett Packard HPL2445w LCD monitor. A general purpose I/O interface 1112 interfaces with a keyboard and/or mouse 1114 as well as a touch screen panel 1116 on or separate from display 1110. General purpose I/O interface 1112 also connects to a variety of peripherals 1118 including printers and scanners, such as an OfficeJet or DeskJet from Hewlett Packard.

A sound controller 1120 is also provided such as Sound Blaster X-Fi Titanium from Creative, to interface with speakers/microphone 1122 thereby providing sounds of alert signals.

The general purpose storage controller 1124 connects the storage medium disk 1104 with communication bus 1126, which may be an ISA, EISA, VESA, PCI, or similar, for interconnecting all of the components of the image processor. A description of the general features and functionality of the display 1110, keyboard and/or mouse 1114, as well as the display controller 1108, storage controller 1124, network controller 1106, sound controller 1120, and general purpose I/O interface 1112 is omitted herein for brevity as these features are known.

In what follows, a detailed description is provided of the fabrication and characterization of medical grade polyurethane composite catheter that can be used for near infrared imaging. According to an embodiment, NIR polymer composites are fabricated into catheters by incorporating a fluorescent dye (IR Dye 800 CW). Specifically, polymer and dye are dry mixed and pressed, sectioned, and further extruded to produce hollow tubes. In order to ensure efficient working of the implanted catheters that include a polyurethane composite, care must be taken that certain characteristics of the composite catheter such as roughness, dye-retention, stiffness, biocompatibility, and near infrared contrast intensity are tested and within acceptable ranges.

According to an embodiment, aromatic polyether-based medical grade thermoplastic polyurethane (TPU) pellets can be mixed with and without IRDye 800CW to form TPU Composite and Plain TPU using a hydraulic platen press. The thermal degradation temperatures are analyzed to verify that both the TPU and IRDye 800CW do not decompose during the extrusion process (described with reference to FIG. 4). The temperature at which the samples begin to decrease sharply in weight is determined to be their degradation point. According to an embodiment, thermal degradation temperatures are evaluated using a Q50 Thermogravimetric Analyzer (TGA), wherein the analysis is conducted in nitrogen gas at 20° C./min (n=3), where n is the number of times the experiment has been performed.

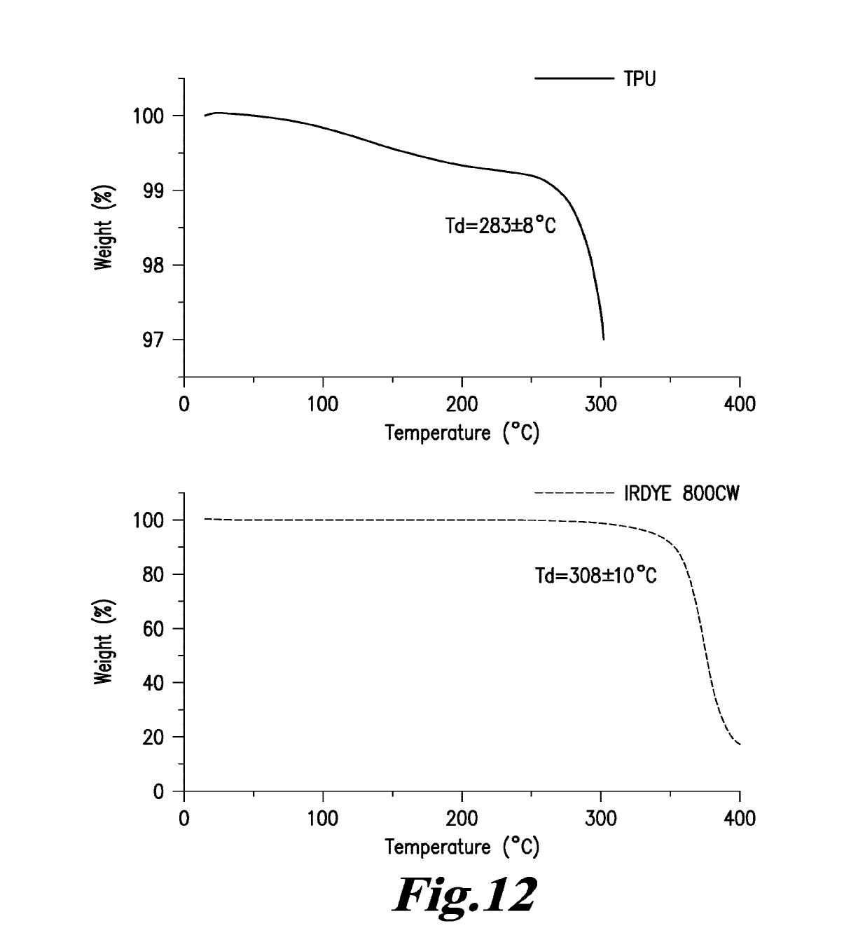

As shown in FIG. 12, TPU and IRDye 800CW display very high degradation temperatures with TPU degrading at 283±8° C. and IRDye 800CW degrading at 308±10° C. Furthermore, the degradation temperatures are considerably higher than the processing temperature of TPU (195° C.).

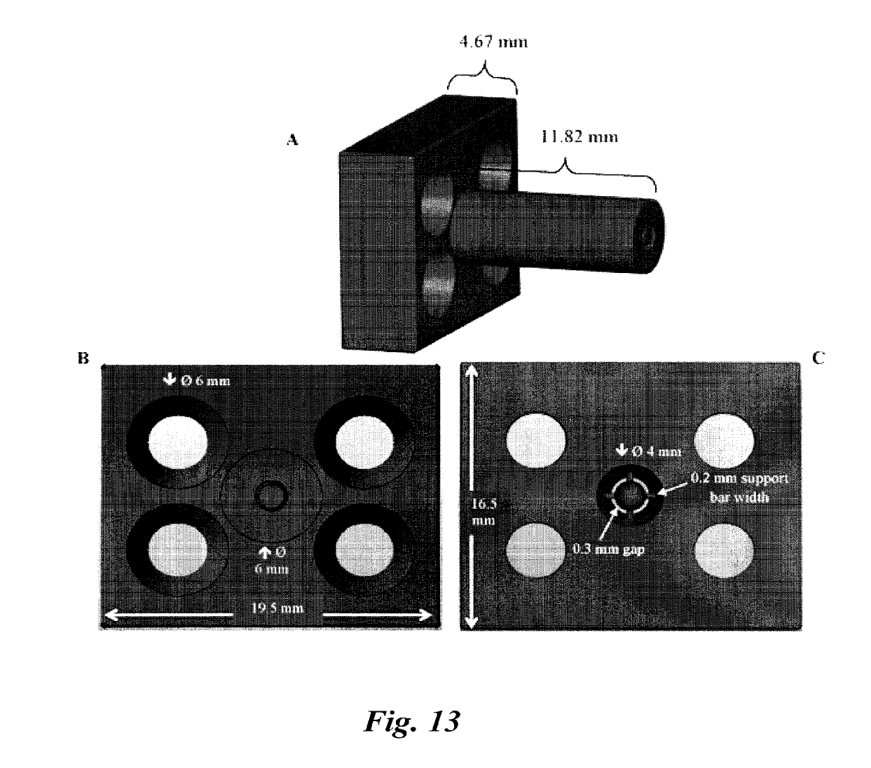

According to another embodiment, a custom annular die as shown in FIG. 13 is fabricated out of stainless steel to produce hollow tubes. In FIG. 13, the figure in part (A) represents a three-dimensional side view with 4.67 mm width and 11.82 mm cylindrical length, through which the TPU is pushed through, figure in part (B) depicts the front view with four 6 mm outer diameter holds which are fastened to the extruder and figure in part (C) depicts the back view of the die showing four support bars which produce the hollow tube feature using a 0.3 mm gap.

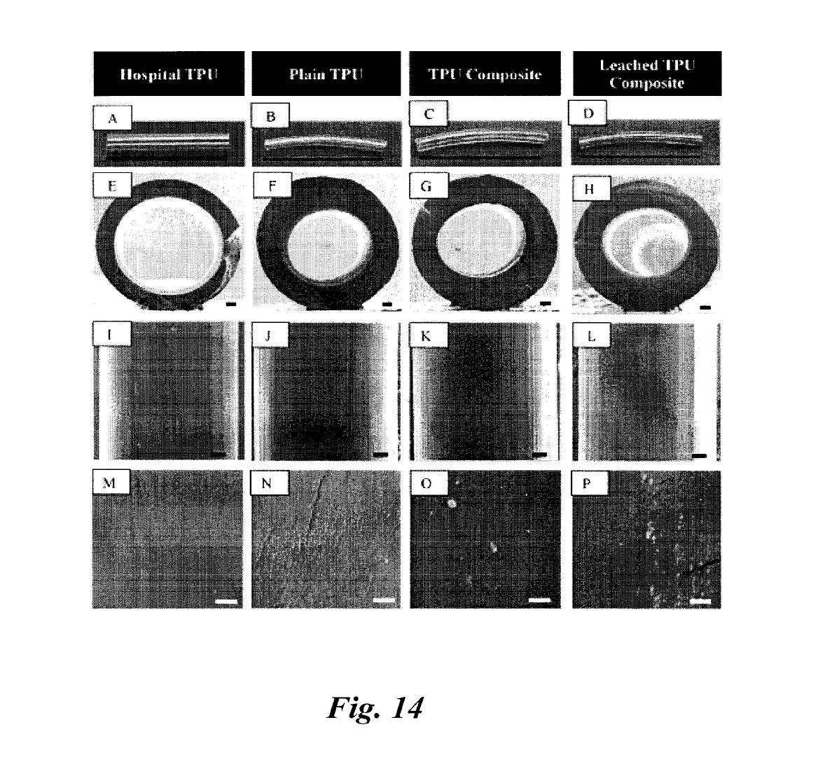

Further, as shown in FIG. 14, the extruded samples are smooth and transparent, and are nearly indistinguishable from a medical grade PICC TPU (referred to herein as Hospital TPU). In FIG. 14, subfigures A-D represent optical images and subfigures E-P depict scanning electron microscopy images (SEM) micrographs of hollow polymer samples. Hospital TPU (subfigure A) is perfectly round and smooth. The extruded samples in subfigure B, C, and D are smooth and optically transparent similar to the Hospital TPU with the composite samples being nearly indistinguishable from their unmodified counterparts. The SEM micrographs include cross sectional views (subfigures E, F, G, and H), top view (subfigures I, J, K, and L), and roughness profiles (subfigures M, N, O, P). Collectively, the extruded samples have larger diameters and thicknesses compared to the Hospital TPU due to the extruder die design.

Further, plain TPU (subfigure F), TPU Composite (subfigure G) and Leached TPU Composite (subfigure H) have irregular cross sectional slices due to swelling and sample collection during extrusion. Top view and roughness images between all samples are similar. Note that in FIG. 14, the optical image scale bar is 1.5 in, whereas the cross sectional and top view scale bar are 200 μm, and the roughness image scale bar is 600 nm.

The TPU composite tubes are slightly darker in color than the unmodified polymer tubes suggesting the fluorescent agent does not significantly alter the appearance of the TPU. Bending of the extruded samples occurs due to the extrusion collection procedure. Extruded samples have an average outer diameters of 2.69±0.11 mm and thickness of 1.65±0.21 mm while the Hospital TPU has an average outer diameter of 2.48±0.11 mm and thickness of 1.19±0.21 mm as shown below in Table 2.

TABLE 2
Outer and Inner Diameters and Thickness Measurements

| | Outer Diameter | Inner Diameter | Thickness |
|---|---|---|---|
| Sample | (mm) | (mm) | (mm) |
| Hospital TPU | 2.48 ± 0.11 | 2.08 ± 0.10 | 1.19 ± 0.21 |
| Plain TPU | 2.74 ± 0.11 | 2.06 ± 0.10 | 1.60 ± 0.21 |
| TPU Composite | 2.74 ± 0.11 | 1.84 ± 0.10 | 1.79 ± 0.21 |
| Leached TPU Composite | 2.57 ± 0.11 | 1.91 ± 0.10 | 1.53 ± 0.21 |

According to another embodiment of the present disclosure, scanning electron microscopy is used to examine the outer surface and cross-sectional features of the catheters. Outer surfaces and cross-sectional features are imaged before and after retention studies of the extruded tubes. Atomic force microscopy is used to obtain quantitative outer surface roughness measurements of the Hospital TPU, Plain TPU, TPU Composite, and Leached TPU Composite. Surface roughness is measured using contact mode of n=3.

Further, tensile testing is performed using an Instron 5500R at a cross head speed of 50 mm/min on Hospital TPU, Plain TPU, TPU Composite, and Leached TPU composite (n=3). To prevent slipping, an Instron clamp with grooved indentations is used. Uniaxial tensile testing is performed on all samples until material failure. The elastic modulus is determined to be the slope from the low strain region (0 to 10%) of the curve, whereas the point of fracture is determined to be the ultimate tensile strength (UTS).

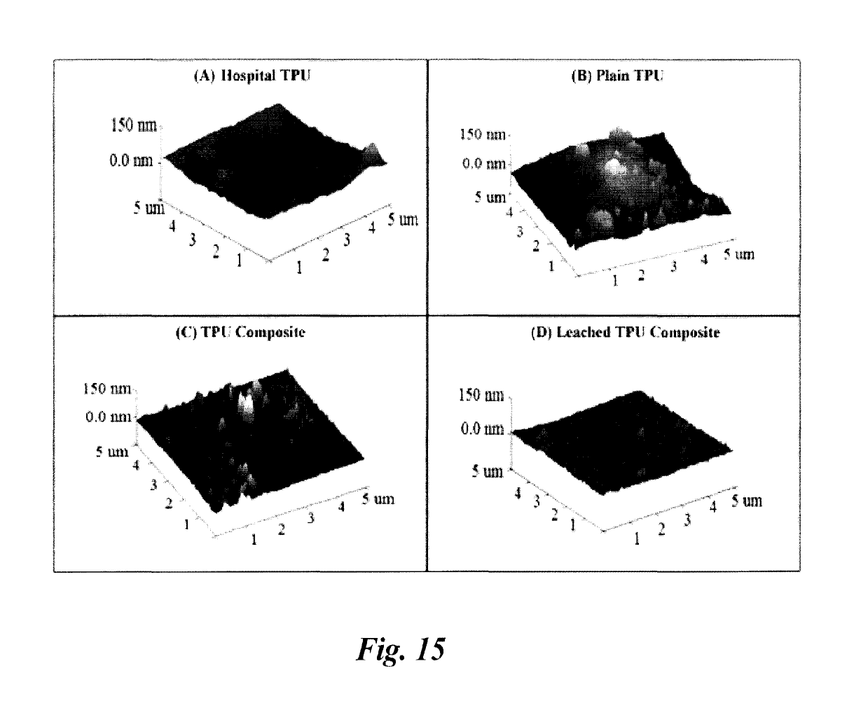

SEM images of extruded samples have irregularly shaped cross sections compared to the circular Hospital TPU as shown in subfigures E-H of FIG. 14. Furthermore, extruded samples are thicker than Hospital TPU. However, surface morphology between extruded samples and Hospital TPU is similar, consisting of defined grain boundaries throughout the microstructure (subfigures I-L of FIG. 14). The TPU composite tubes contain light precipitates dispersed throughout the polymer surface demonstrating the presence of fluorescent agent (as shown in subfigures O and P of FIG. 14). Furthermore, quantitative roughness measurements, shown in Table 3 below, that are obtained from AFM contact mode revealed that Hospital TPU has the smoothest surface while Plain TPU contains roughness values that are statistically significant compared to all other samples (p<0.05). No statistical significance in roughness exists between TPU Composite and Leached TPU Composite tubes as compared to Hospital TPU, suggesting the addition of fluorescent agent does not significantly alter roughness morphology. Furthermore, the mixing of the fluorescent dye with TPU acts as a plasticizer, smoothing rough areas during the extrusion process as is evidenced by the increased roughness in Plain TPU samples as depicted in FIG. 15.

TABLE 3
TPU roughness (Ra) measurements

| Sample | AVG Ra (nm) |
|---|---|
| Hospital TPU | 4.86 ± 1.38 |
| Plain TPU | 19.07 ± 7.36 |
| TPU Composite | 7.34 ± 1.78 |
| Leached TPU Composite | 6.52 ± 2.42 |

In FIG. 15, a 5 μm×5 μm area is scanned using contact mode. While the Plain TPU is statistically rougher than all the other samples, the roughness profiles of the TPU Composite and Leached TPU Composite samples is not statistically different compared to the Hospital TPU. However, note that Plain TPU roughness measurements are significantly different compared to the other samples (p<0.05).

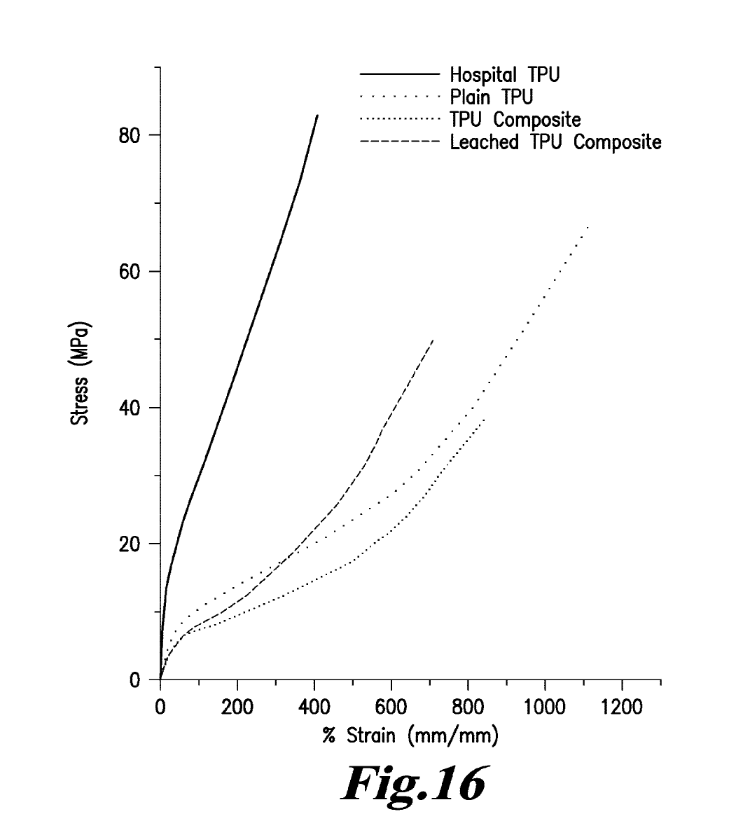

During tensile testing, samples that slipped before failure are not included in data analysis. Failure occurred at the clamped ends of all samples. Hospital TPU has the highest average elastic modulus (1.87±0.19 MPa), while TPU Composite has the lowest elastic modulus (0.17±0.005 MPa) as shown below in Table 4 and illustrated in FIG. 16.

TABLE 4
Mechanical Property Measurements of Samples

| | AVG Elastic Modulus | |
|---|---|---|
| Sample | (MPa) | AVG UTS (MPa) |
| Hospital TPU | 1.87 ± 0.19 | 88.1 ± 8.58 |
| Plain TPU | 0.19 ± 0.02 | 56.4 ± 17.6 |
| TPU Composite | 0.17 ± 0.005 | 50.0 ± 10.3 |
| Leached TPU Composite | 0.23 ± 0.03 | 62.22 ± 19.6 |

Although the Hospital TPU elastic modulus and ultimate tensile strength (UTS) are significantly different compared to the extruded samples, there is no statistical difference within the extruded samples, suggesting that the addition of IRDye 800CW does not alter the mechanical properties of the TPU.

According to another embodiment of the disclosure, in order to determine the long-term effect of being implanted in vivo, catheters are leached in phosphate buffered saline powder (PBS) for 23 days to determine the amount of dye retained within the matrix. TPU Composite tubes are cut into thin slices, weighed, and added to a black 96 well plate containing 200 μI PBS. Leaching of IRDye 800CW from the TPU Composite (n=8) is analyzed under physiological conditions (pH ˜7.4, 37° C., with gentle agitation) in a water bath. The water bath is covered to prevent photo-bleaching. Each day, tubes can be transferred to the successive well containing PBS, and the previous day is analyzed using a micro-plate reader with excitation at 765 nm, emission at 794 nm and a sensitivity of 100. Note that the wavelengths do not represent peak emission and excitation wavelengths, but are wavelengths of sufficient magnitude to perform near infrared imaging. Further, to determine the amount of IRDye 800CW retained, a calibration curve containing serial dilutions of IRDye 800CW in PBS is used (0 to 0.00030 wt %) (R²=0.99). Additionally, 10 mL of IRDye 800CW in PBS is placed in the water bath and 100 μL aliquots are analyzed per day for signs of signal degradation due to heat.

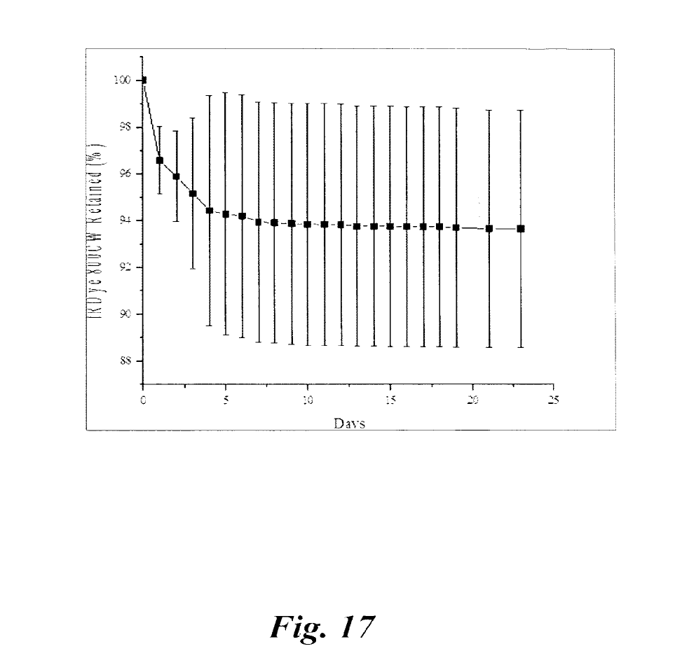

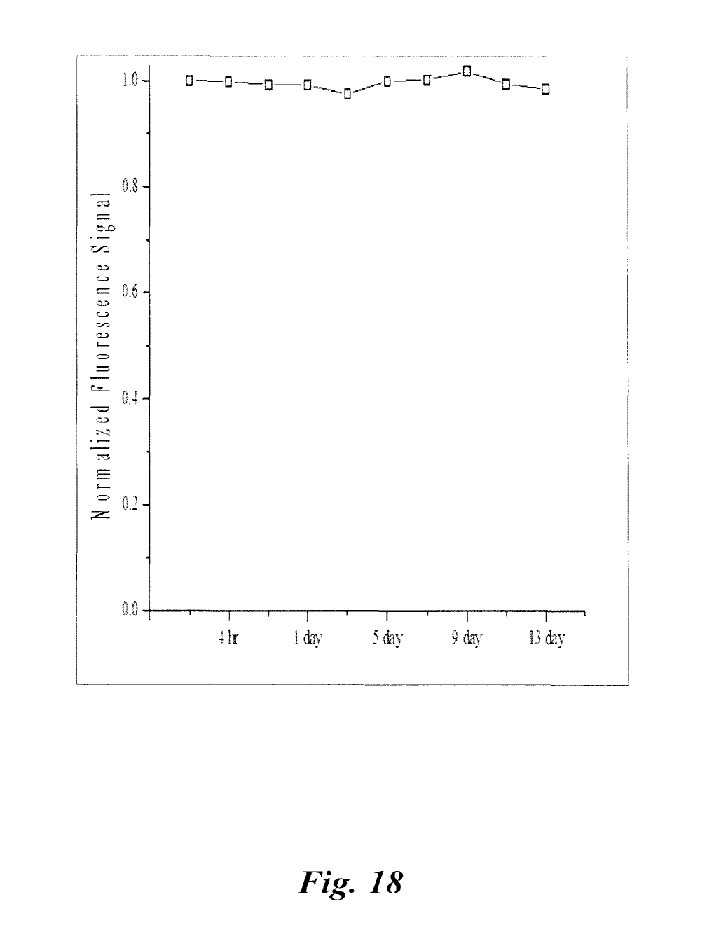

As shown in FIG. 17, daily analysis of PBS from TPU Composite tubes incurs a total loss of 6.35±5.08% from within the polymer matrix over a 23-day period. The retention analysis of IR Dye 800 CW within TPU matrix of FIG. 17 includes a 6.35% of the IR Dye 800 CW being released from the polymer over 23 days. The majority of the dye is released as a burst within the first five days and approximately 5.40% follows minimal leaching throughout the duration of the study. A control of IRDye 800CW in PBS was also maintained in the water bath to determine if physiological conditions cause degradation of the fluorescent signal. FIG. 18 depicts the stability of IRDye 800CW in PBS at 37° C. with gentle agitation. As shown in FIG. 18, there is no observed decrease in the fluorescent signal over a period of 12 days.

According to another embodiment of the present disclosure, photo-degradation and fluorescent imaging analysis is performed of the PICC implanted device. Specifically, in order to determine the contrast enhancement due to the addition of IRDye 800CW, samples are imaged on a LI-COR Pearl Impulse NIR imaging system and analysis is performed in LI-COR Pearl Impulse Software. Shapes are drawn manually around the samples and the signal-to-noise ratio (SNR) is computed (Mean of Sample/Standard deviation of Background). To determine the optimal loading concentration, thin films of TPU containing 0.025, 0.075 and 0.125 wt % IRDye 800CW are pressed and imaged. Hydration effects of Plain TPU and TPU Composite tubes are analyzed by imaging dry, 24 hour PBS soaked then dried, and hydrated samples (in PBS) with the LI-COR System. For investigation of photo-bleaching, TPU Composite tubes are placed 6 inches beneath a 13-watt halogen light source for ten days. Samples are removed daily for fluorescence intensity analysis. The error bars represent variation within a sample wherein a signal is calculated at each pixel within the sample (n=1). Further, to determine whether the fluorescent signal degrades due to repeated imaging with the LICOR system, samples are imaged 20 consecutive times and fluorescent intensities are compared.

Additionally, in order to determine contrast enhancement of the TPU Composite tubes, samples are hydrated for 24 hours in PBS to simulate physiological conditions, placed in the LI-COR system without Superflab® tissue mimic and imaged to acquire the 0 cm fluorescence intensity. Imaging is repeated with 1, 2, 3, and 4 cm of Superflab placed over the samples. Images are analyzed by automatic shape drawing around each sample in the 0 cm image. The shapes are copied to successive images containing Superflab®, and SNR is calculated for each. Enhancement factors are calculated by dividing the SNR of TPU Composite by the SNR of Plain TPU. Standard deviations are computed from SNR of the four samples and scaled by the background noise.

According to an embodiment, the optimal loading level of IRDye 800CW is 0.025 wt %. Concentrations greater than 0.025 wt % result in quenching of the fluorescent signal as depicted in FIG. 16 and shown below in Table 5.

TABLE 5
Composite Thin Film Intensity Measurements

| IRDye 800CW (wt %) | SNR |
|---|---|
| 0.025 | 224 |
| 0.075 | 60 |
| 0.125 | 31 |

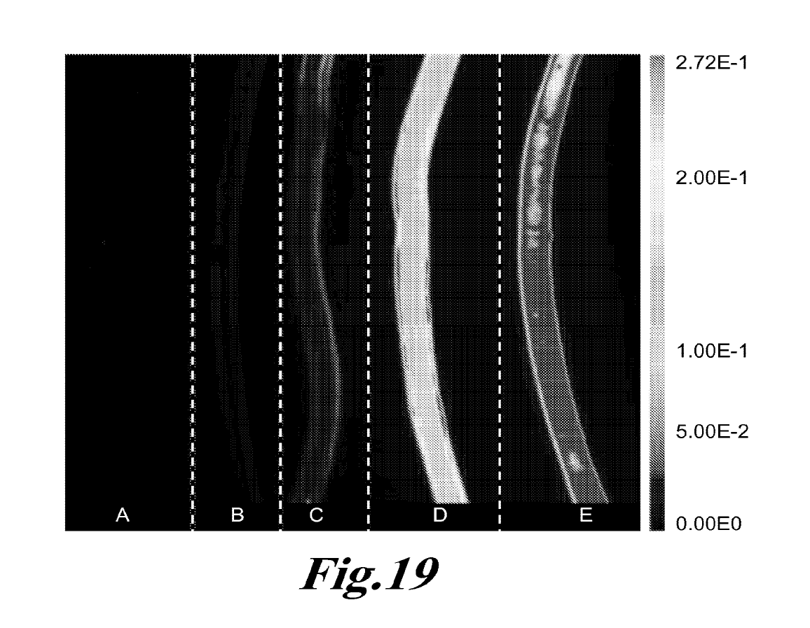

Fluorescent signal increases significantly if the TPU Composite is soaked in PBS for 24 hours and dried prior to imaging as illustrated in FIG. 17 and shown below in Table 6. Further enhancement of signal intensity occurs when the TPU Composite is completely hydrated in PBS compared to the dry state (as depicted in FIG. 19).

TABLE 6
TPU Intensity Scan Descriptions

| Tube | Descriptions |
|---|---|
| A | TPU Always Dry |
| B | TPU Soaked in PBS and Dried |
| C | TPU Composite Always Dry |
| D | TPU Composite Soaked then Dried |
| E | TPU Composite in PBS |

FIG. 19 depicts computer aided design schematic of annular dye. In FIG. 19, region (A) depicts a three-dimensional side view with 4.67 mm width and 11.82 mm cylindrical length, which the TPU is pushed through. Region (B) is a front view with four 6 mm outer diameter holds which are fastened to the extruder. Region (C) is back view of the die showing four support bars which produce the hollow tube feature using a 0.3 mm gap. Further, photo-degradation studies show no significant loss of signal over a 10-day period. Substantial variation in the SNR is observed which is due to changes in radius of the extruded samples. Furthermore, repeated imaging studies revealed no loss in signal when samples are imaged multiple times.

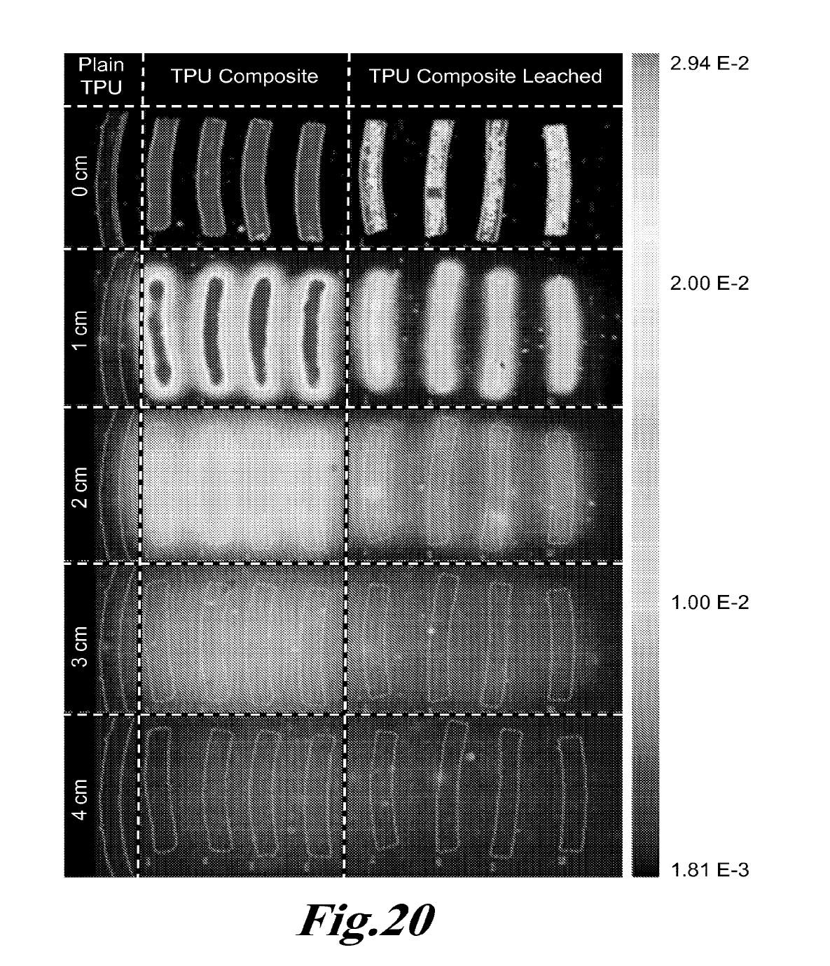

Additionally, fluorescent scans of the TPU Composite tubes result in a 14-fold increase in SNR as compared to the Plain TPU tubes. Such a contrast enhancement allows imaging of the extruded tubes up to depths of 4 cm, as shown in FIG. 20. In FIG. 20, samples are imaged at an excitation wavelength of 778 nm. Further, the numbers depicted in the right hand portion of FIG. 20, i.e., 0, 1, 2, 3, and 4 cm correspond to the imaging depth or the thickness of Superflab covering the samples that the imaging probe penetrated.

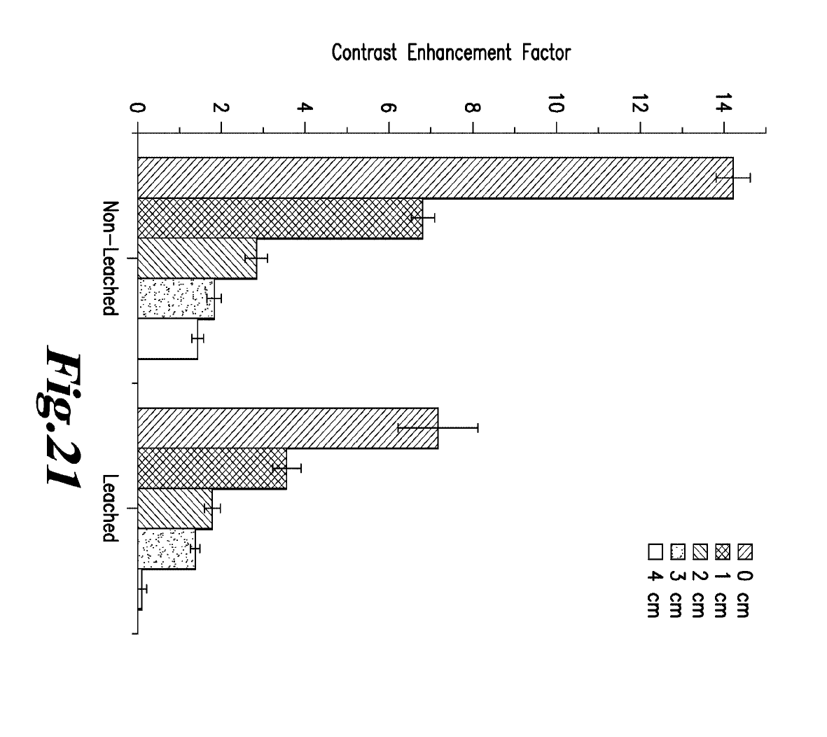

A 50% reduction in signal is observed between the leached and non-leached samples. Non-leached and leached samples are significantly different at every depth (p<0.05) while there was no statistical difference within either group at 3 and 4 cm. A 50% reduction in signal was observed between the leached and non-leached samples. Non-leached and leached samples were significantly different at every depth (p<0.05) while there was no statistical difference within either group at 3 and 4 cm as shown in FIG. 21. Note that the fluorescence intensity decreases as a function of depth, though signal is still observed at 4 cm. All values are statistically significant both within and between the non-leached and leached samples except within 3 cm and 4 cm.

According to another embodiment of disclosure, biocompatibility studies are conducted to determine the toxicity of TPU Composite in direct contact with endothelial cells as well as the adhesion of endothelial cells to the TPU Composite. Pressed films (Plain TPU and TPU Composite) are sterilized by washing in 1×PBS for 24 hours under constant agitation, followed by a 30 minute soak in 100% ethanol and two 1 hour rinses with PBS. Biocompatibility studies include 12 well cell bind plates being seeded with Human Umbilical Vein Endothelial (HUVEC) cells (passage 4-10, cultured in complete endothelial growth medium EGM Bulletkit, LONZA) at a density of 100 cells/cm² for 12 hours to allow for adhesion [37° C., 5% CO₂]. Films (19 mm) are placed in direct contact to the cells and incubated for an additional 72 hours, replacing media daily. Toxicity is quantitatively analyzed with alamar blue according to manufacturer's protocol. Briefly, 100 μL of alamar blue is added to the media and allowed to incubate for 1.5 hours. Fluorescence of each alamar blue was read at excitation 545 nm, emission 590 nm. Films are removed from wells and cells are stained with Calcein AM and propidium iodide according to manufacturer's protocol, fixed with 4% paraformaldehyde for 1 hour and rinsed three times with PBS for qualitative analysis of cell death. Cells are imaged with fluorescent confocal imaging (Zeiss) for proliferation, morphology changes and viability. The process is repeated with 0.025 wt % IRDye 800CW in media, cells with media as a positive control, and cells with 70% ethanol in media as a negative control.

To determine if endothelial cells bind to the catheters, 19 mm films are cut and affixed to the bottom of suspension 12-well culture plates with 50 μL of 10 mg/mL collagen Type I isolated from rat tails. Plates are incubated for 30 minutes to allow for collagen polymerization. Films are seeded with 100 cells/cm² and incubated for one hour. Wells are washed with PBS to remove non-adherent cells and stained with Calcein AM and propidium iodide to aid in visualization of cell binding. The number and cell health of adherent cells is analyzed with fluorescence microscopy and compared to positive (collagen plates) and negative (Teflon) controls.

After a 72 hour incubation of HUVECs with IRDye 800CW (0.025 wt %), Plain TPU and TPU Composite, no statistical difference is observed in cell viability as shown in Table 7.

TABLE 7
Biocompatibility Results

| Sample | Normalized Viability |
|---|---|
| Media | 100 ± 5.13 |
| Plain TPU | 94.79 ± 3.26 |
| TPU Composite | 92.34 ± 3.93 |
| 0.025 wt % IRDye 800CW | 91.27 ± 8.54 |

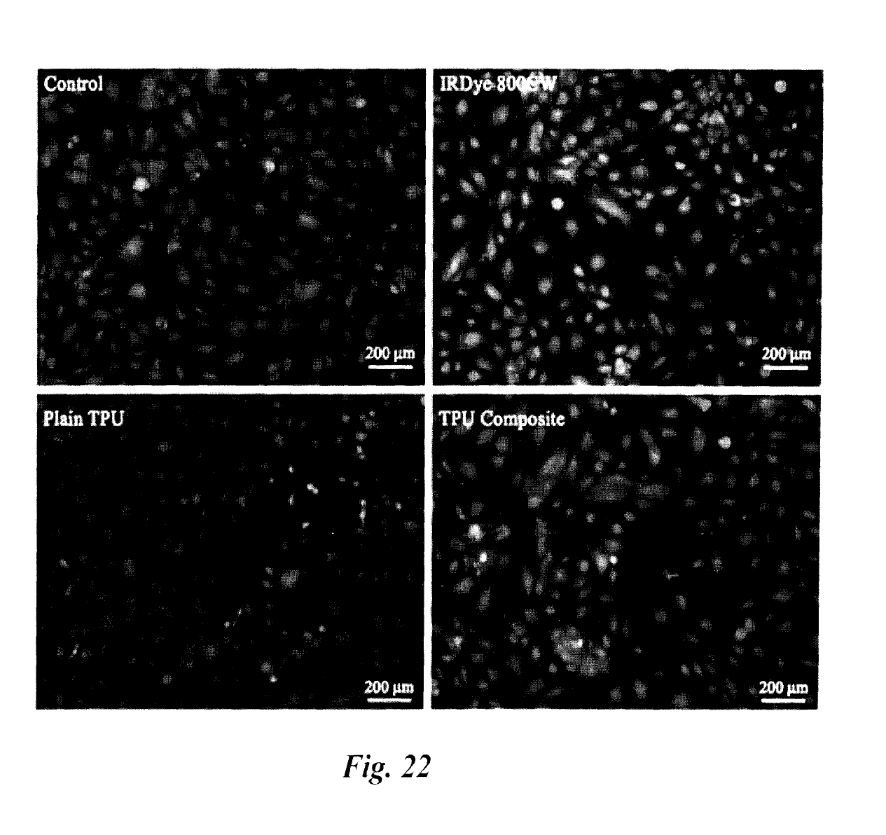

Viability values are normalized to the media control values. The results are confirmed with Calcein AM and Propidium Iodide Staining as shown in FIG. 22. The majority of cells are viable, and no apparent change in cell morphology or proliferation rates is observed due to the IRDye 800CW, Plain TPU or TPU Composite.

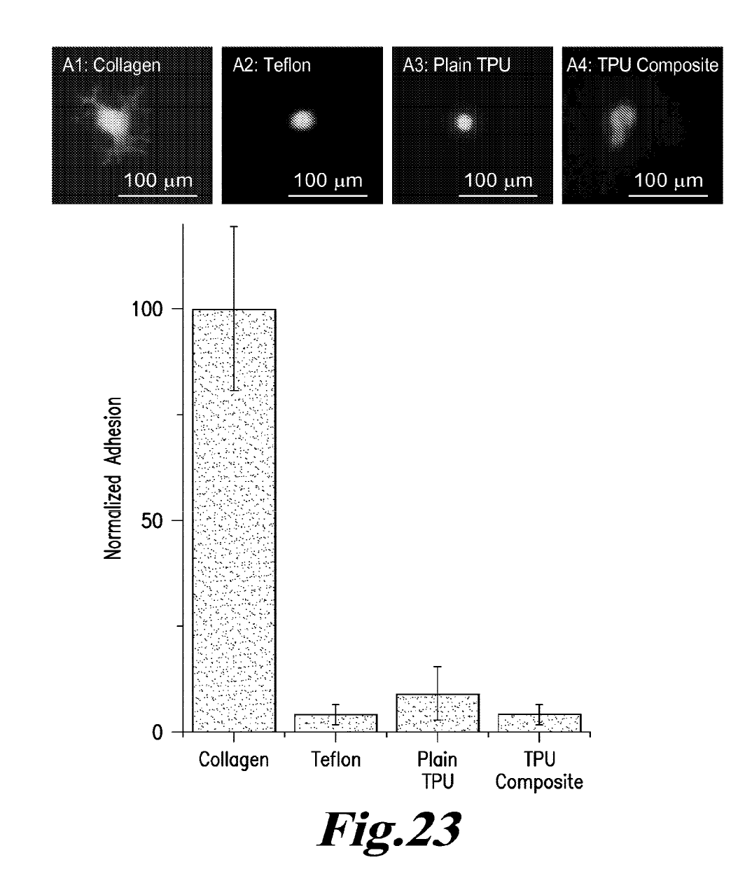

In order to be a viable biomaterial, cell adhesion should be minimal in order to avoid excess damage when removing or inserting the PICC. Cells preferentially adhered to Collagen I, a protein found in the native microenvironment of the extracellular matrix. The cells increase substantially in area due to spreading with extended lamillopodia demonstrating their affinity for the material (as shown in FIG. 23, A1). The rounded shape of the cells with no extended protrusions, indicate weak adherence to the negative control (Teflon) as well as the Plain TPU and TPU Composite (FIG. 23, A2-A4). The number of adhered cells is counted using ImageJ particle analyzer software (NIH) from 6 images and normalized to Collagen I. Cell adherence to Teflon, Plain TPU and TPU Composite are significantly different from Collagen I but are not significantly different between each other (as shown in FIG. 23, portion B).

Embodiments described herein have many applications which allow imaging of a wide variety of medical devices implanted inside a living body. Peripherally-inserted central catheters, which are fluorescent-coated or made with fluorescent-impregnated material, can be imaged and monitored after placement. Other applications include, but are not limited to, imaging of cardiac implants, joint surfaces, and endotracheal tubes.

Furthermore, the monitoring of implanted catheters can also be performed by photoacoustic imaging techniques. Photoacoustic imaging is a hybrid biomedical imaging modality that is based on the photo-acoustic effect. In photoacoustic imaging, non-ionizing laser pulses are delivered into biological tissues. Some of the delivered energy is absorbed and converted into heat, leading to transient thermo-elastic expansion and thus wideband (e.g. MHz) ultrasonic emission. The generated ultrasonic waves can be detected by ultrasonic transducers to form photoacoustic images of the fluorescent catheter (or any other medical implant). The standard B-mode ultrasound imaging method can also be used to form a gray-level image of the anatomy hosting the catheter (or any other implant). The combined photoacoustic and gray-level ultrasound can show the exact location of the catheter with respect to the surrounding anatomy. The combined image can also help monitor the potential migration of the implanted catheter.

For instance, a device can be irradiated with NIR light and emit, in return, NIR light of a lower energy. This lower energy NIR light is detected by an imager and allows for the device to be located. In contrast, in photoacoustic imaging, the device is irradiated with, for example but not limited to, high intensity NIR light and this causes a reaction in the near infrared (NIR) dye as well as a distortion in the immediate surroundings. This reaction is a result of absorption by the infrared NIR dye. These reactions and distortions are detected by ultrasound and thereby allow for the device to be located. Thus, ultrasound can be used in lieu or in supplement of the NIR sensitive imager. Photoacoustic imaging can be used to locate NIR devices irradiated by NIR light with ultrasound techniques. Moreover the ultrasound techniques can also provide images of surrounding organs, vessels, and various tissue structures during placement of the medical device.

While aspects of the present disclosure have been described in conjunction with the specific embodiments thereof that are proposed as examples, alternatives, modifications, and variations to the examples may be made. Accordingly, embodiments as set forth herein are intended to be illustrative and not limiting. There are changes that may be made without departing from the scope of the claims set forth below.

1-5. (canceled)

6. A neonatal, peripherally-inserted central catheter (PICC), comprising:
a hollow, polymeric, flexible tube having a distal tip, the tube having a length at least long enough for percutaneous, intravascular positioning of the distal tip proximate the heart of a neonatal patient; and
a fluorescent dye comprising a fluorophore configured to absorb near-infrared (NIR) light and to emit light at a different wavelength than the absorbed light;
wherein the fluorescent dye is located on at least a portion of the catheter that is configured for positioning within the vascular system of the patient.

7. The neonatal PICC of claim 6, wherein the fluorescent dye is coated on at least a portion of the catheter.

8. The neonatal PICC of claim 7, wherein the fluorescent dye is coated on the full length of the catheter.

9. The neonatal PICC of claim 6, wherein at least a portion of the polymeric tube is impregnated with the fluorescent dye.

10. The neonatal PICC of claim 9, wherein the full length of the polymeric tube is impregnated with the fluorescent dye.

11. The neonatal PICC of claim 6, wherein the fluorophore emits light after absorption of light between 650 nm and 950 nm in wavelength.

12. A system comprising:
a. the neonatal PICC of claim 6; wherein the system further comprises:
b. an excitation light source, positioned external to the neonatal patient, and configured to emit light at a wavelength in the near-infrared band of the electromagnetic wave spectrum for penetration of the neonatal patient and for absorption by the fluorophore when the distal catheter tip is located in the vasculature proximate the heart of the neonatal patient; and
c. a light detector, positioned external to the neonatal patient, and configured to detect light emitted from the fluorophore and to convert the detected light into a measurable electrical signal for conversion into an image.

13. The system of claim 12, further comprising at least one processor configured to generate an image of at least a portion of the catheter from the light detected by the light detector.

14. The system of claim 13, wherein the processor is further configured to store at least one generated image and to compare one or more subsequently generated images to the stored image.

15. The system of claim 14, wherein the processor is further configured to compute a deviation of the position of the catheter within the neonatal patient by comparing two or more generated images.

16. The system of claim 13, further comprising a marker configured for placement on the skin of the neonatal patient.

17. The system of claim 16, wherein the marker is located on a patch for affixing to the skin of the neonatal patient.

18. The system of claim 16, wherein the marker comprises a fluorophore configured to absorb near-infrared (NIR) light and to emit light at a different wavelength than the absorbed light.

19. The system of claim 18, wherein the excitation light source is configured to excite the fluorophore in the marker and of the fluorescent dye.

20. The system of claim 19, wherein the light detector is configured to detect light emitted from the fluorophore in the marker and from the fluorescent dye.

21. The system of claim 20, wherein the processor is further configured to compare the location of at least a portion of the catheter to the position of the marker.

22. A method of detecting the position of a neonatal peripherally-inserted central catheter in the vasculature of a neonatal patient, comprising:
a. percutaneously inserting and advancing the neonatal PICC of claim 6 intravascularly until the distal tip is proximate the heart of the neonatal patient;
b. directing NIR light into the neonatal patient from a position external to the neonatal patient to excite the fluorophore of the fluorescent dye; and
c. detecting light emitted from the fluorophore with a detector located external to the neonatal patient, wherein the detected light is used to detect the position of the neonatal PICC in the neonatal patient.

23. The method of claim 22, further comprising generating an image of at least a portion of the neonatal PICC from light emitted from the fluorophore.

24. The method of claim 23, further comprising generating at least one subsequent image of a least a portion of the neonatal PICC from light emitted from the fluorophore.

25. The method of claim 24, further comprising comparing at least two of the generated images to determine movement of the catheter within the neonatal subject vasculature.

26. The method of claim 22, further comprising:
a. affixing a marker to the skin of the neonatal patient; and
b. determining the position the affixed marker relative to at least a portion of the neonatal PICC when the neonatal PICC is positioned within the neonatal patient vasculature.

27. The method of claim 26, wherein the relative position of the portion of the neonatal PICC compared to the affixed marker is used to assess movement of the catheter within the neonatal patient vasculature.
